(19) United States
(12) Patent Application Publication
(10) Pub. No.: US 2011/0034856 A1
(43) Pub. Date: Feb. 10, 2011

(54) DEVICE AND METHOD FOR ENHANCED WOUND TREATMENT

(76) Inventor: Yaakov BREZEL, Jerusalem (IL)

Correspondence Address: Marc Van Dyke, 123 N.W. 13th Street, Suite 221, Boca Raton, FL, 33432-1619, US

(21) Appl. No.: 12/853,491

(22) Filed: Aug. 10, 2010

Related U.S. Application Data
(63) Continuation-in-part of application No. PCT/IL2009/000145, filed on Feb. 8, 2009.

(51) Int. Cl. A61N 1/36; A61M 1/00

(52) U.S. Cl. 604/20

(57) ABSTRACT

An apparatus for facilitating the healing of a wound on a limb of a body of a subject, including: (a) a wound treatment assembly having: (i) a sealing arrangement adapted to cover an area above the wound, and to contact and at least partially seal a volume from an ambient environment, and (ii) a vacuum mechanism fluidly communicating with the volume, and adapted to produce a sub-atmospheric pressure therein; (b) a muscle contraction device having at least first and second electrodes, each adapted to operatively contact the limb; (c) a control unit, adapted to connect to a power supply and operatively connected to each electrode, and further adapted to provide, via the electrodes, a sequence of electrical impulses to points associated with the limb, whereby muscle tissue associated with the points contracts to effect a localized increase in afferent and efferent flow of blood through blood vessels in the limb, the control unit being operatively connected to the assembly and further adapted to control an operation thereof.

### CROSS-REFERENCE TO RELATED APPLICATIONS

This application is a continuation-in-part (CIP) application of PCT/IL2009/000584 filed on Feb. 8, 2009 and claims priority to U.S. Provisional Patent Application Ser. No. 61/027,464, filed on Feb. 10, 2008, both of which are hereby incorporated by reference for all purposes as if fully set forth herein.

### FIELD AND BACKGROUND OF THE INVENTION

The present invention relates to a method and device for treating a wound by means of a sub-atmospheric pressure applied in the vicinity of the wound, and more particularly, to a treatment method and device in which sub-atmospheric pressure therapy is effected in conjunction with a method and device for contracting muscles to locally increase the flow of blood in that vicinity.

It is generally accepted that such sub-atmospheric-based treatments are indicated only for patients who have a reasonably healthy local flow of blood in the vicinity of the wound to be treated. One assessment used to evaluate the peripheral blood flow in the lower extremities is the ankle-brachial index (ABI). This test uses a Doppler instrument to measure blood pressure at arteries of both the ankle (either the Dorsalis Pedis or Posterior Tibial arteries) and the arm (Brachial artery) while a person is at rest. The result of the arm pressure (in mmHg) divided by the ankle pressure (in mmHg) provides the ABI. This assessment tool is based on the understanding that in healthy individuals, the blood flow in the upper and lower extremities should be similar, and that as peripheral arterial disease worsens, the blood flow in the legs decreases comparatively. The ankle-brachial index (ABI) result may be used to predict the severity of peripheral arterial disease (PAD).

A resting ankle-brachial index of greater than 0.9 may be generally considered to be normal and indicates that there is no significant narrowing or blockage of blood flow in the lower extremities.

If the ABI is less than 0.9, narrowing of one or more blood vessels in the legs and thus, reduction of blood flow is likely. If the ABI is less than 0.8, pain in the foot, leg, or buttock may occur during exercise (intermittent claudication). If the ABI is less than 0.4, various symptoms may occur such as an aching, tired, or burning pain in the legs, even when the patient is at rest. If the ABI is less than 0.25 or below, severe limb-threatening PAD may manifest as open wounds, cyanosis or gangrenous changes of the tissues of the foot and leg.

Certain conditions including diabetes or renal insufficiency may result in progressive calcification of the arteries more than simple arterial disease alone.

It is imperative to identify the presence or absence of PAD via the ABI as well as the blood pressure in the leg itself, as a low ABI (e.g., below about 0.7) or the blood pressure itself in a close range to the intended pressures of the treatment may be a clear contra-indication for using sub-atmospheric pressure therapy. For a vast number of patients having a low ABI index, such sub-atmospheric pressure therapy may be largely ineffective, and may further compromise an already tenuous blood flow. It is believed that there is room and need for further improvements in methods and devices for treating a wound using sub-atmospheric pressure, and the subject matter of the present disclosure and claims is aimed at fulfilling this need.

### SUMMARY OF THE INVENTION

According to the teachings of the present invention there is provided an apparatus for facilitating the healing of a wound on a limb of a body of a subject, the apparatus including: (a) a wound treatment assembly including: (i) a wound cover adapted to cover an area above the wound; (ii) a sealing arrangement, associated with the cover, adapted to contact and at least partially seal a volume beneath the cover from an ambient environment; (iii) a vacuum mechanism fluidly communicating with the volume, and adapted to produce a sub-atmospheric pressure between about 0.01 (about 75 mmHg) and 0.99 bar absolute (about 742 mmHg), more typically, between about 0.03 bar and 0.3 bar absolute, and yet more typically, between 0.05 and 0.25 bar absolute, within the volume; (b) a muscle contraction device having at least a first electrode and a second electrode, each electrode adapted to operatively contact the limb; (c) a control unit, adapted to connect to a power supply and operatively connected to each electrode, the control unit further adapted to provide, via the electrodes, a sequence of electrical impulses to neural points associated with the limb, whereby muscle tissue associated with the neural points contracts to effect a localized increase in a flow of blood through a blood vessel in the limb, the control unit operatively connected to the wound treatment assembly and further adapted to control an operation of the treatment assembly.

According to another aspect of the present invention there is provided an apparatus for facilitating the healing of a wound on a limb of a body of a subject, including: (a) a wound treatment assembly including: (i) a sealing arrangement adapted to cover an area above the wound, and to contact and at least partially seal a volume from an ambient environment, and (ii) a vacuum mechanism fluidly communicating with the volume, and adapted to produce a sub-atmospheric pressure within the volume; (b) a muscle contraction device having at least a first electrode and a second electrode, each electrode adapted to operatively contact the limb; (c) a control unit, adapted to connect to a power supply and operatively connected to each electrode, the control unit further adapted to provide, via the electrodes, a sequence of electrical impulses to points associated with the limb, whereby muscle tissue associated with the points contracts to effect a localized increase in a flow of blood through a blood vessel in the limb, the control unit operatively connected to the wound treatment assembly and further adapted to control an operation of the treatment assembly.

According to yet another aspect of the present invention there is provided a method of facilitating the healing of a wound on a limb, including the steps of: disposing a wound cover over the wound; contacting a sealing arrangement with skin surrounding the wound; activating a vacuum mechanism to produce a sub-atmospheric pressure within the volume, and (e) delivering a sequence of electrical impulses, via the electrodes, to effect a localized change in a flow of blood through a blood vessel in the limb.

According to further features in the described preferred embodiments, the control unit has at least a first operating mode enabling a combined treatment protocol including both operation of the wound treatment assembly and operation of the muscle contraction device.

According to still further features in the described preferred embodiments, the control unit is configured to implement a combined treatment protocol including simultaneous operation of the wound treatment assembly and the muscle contraction device.

According to still further features in the described preferred embodiments, the control unit is configured to implement a combined treatment protocol including at least intermittent operation of both the wound treatment assembly and the muscle contraction device.

According to still further features in the described preferred embodiments, the control unit is configured to implement the combined treatment protocol responsive to a pre-determined sequencing.

According to still further features in the described preferred embodiments, the control unit is configured to receive the pre-determined sequencing via an input unit associated with the control unit.

According to still further features in the described preferred embodiments, the control unit has at least a second operating mode enabling a treatment protocol including solely operation of the wound treatment assembly.

According to still further features in the described preferred embodiments, the control unit has at least a second operating mode enabling a treatment protocol including solely operation of the muscle contraction device.

According to still further features in the described preferred embodiments, the apparatus further includes a mode selection switch, associated with the control unit, adapted to select between the first and second operating modes.

According to still further features in the described preferred embodiments, the control unit is configured to prompt a user for an ankle-brachial index (ABI) of the subject.

According to still further features in the described preferred embodiments, the control unit is disposed in a single housing.

According to still further features in the described preferred embodiments, the control unit is configured to perform at least one safety operation responsive to an ABI and/or an ankle pressure below pre-determined values.

According to still further features in the described preferred embodiments, the safety operation includes producing a warning signal.

According to still further features in the described preferred embodiments, the safety operation includes displaying a recommended treatment protocol.

According to still further features in the described preferred embodiments, the safety operation includes disabling an option of operating solely the wound treatment assembly.

According to still further features in the described preferred embodiments, the operation of the treatment assembly includes a depth of vacuum produced by the vacuum mechanism.

According to still further features in the described preferred embodiments, the apparatus further includes a measurement unit adapted to produce at least one measurement of a parameter associated with blood flow in the subject, the control unit being configured to perform at least one safety operation responsive to the measurement.

According to still further features in the described preferred embodiments, the wound cover includes a screen, disposed within the volume, the screen being adapted to prevent overgrowth of tissue in the wound.

According to still further features in the described preferred embodiments, the screen includes an open-cell polymer foam.

According to still further features in the described preferred embodiments, the sealing arrangement includes a flexible sealing rim adapted to sealably contact skin surrounding the wound.

According to still further features in the described preferred embodiments, the sealing arrangement includes a flexible polymer sheet, the polymer sheet having an adhesive on at least one surface to attach and seal the polymer sheet to skin surrounding the wound.

According to still further features in the described preferred embodiments, the sealing arrangement includes a flexible polymer sheet overlying the screen, the polymer sheet having an adhesive on at least one surface to attach and seal the polymer sheet to skin surrounding the wound.

According to still further features in the described preferred embodiments, the sealing arrangement includes a sealing cuff in contact with skin surrounding the wound.

According to still further features in the described preferred embodiments, the control unit is adapted to produce the electrical impulses of a magnitude and frequency to effect a series of individual, substantially continuous muscle contractions.

According to still further features in the described preferred embodiments, the control unit includes a signal generator adapted to produce the electrical impulses to effect a series of muscle contractions.

According to still further features in the described preferred embodiments, the muscle tissue is stimulated to effect improved venous flow within the limb.

According to still further features in the described preferred embodiments, the muscle tissue is stimulated to augment the local arterial flow within the limb.

According to still further features in the described preferred embodiments, the muscle tissue is stimulated to promote improved flow of lymphatic drainage in the muscle tissue. The overall effect of these is to improve overall circulatory status in the efferent and afferent circulatory systems, thereby producing an overall localized increase in the flow of blood.

According to still further features in the described preferred embodiments, the method further includes the steps of: (f) providing an ankle-brachial index (ABI) of the subject, and/or a blood pressure of the affected lower limb, and (g) responsive to the ABI and/or this blood pressure, controlling the apparatus to treat the subject.

According to still further features in the described preferred embodiments, the method further includes the steps of: (f) providing the control unit with an ankle-brachial index (ABI) of the subject, and/or a blood pressure of the affected lower limb, and (g) responsive to the ABI and/or this blood pressure, controlling the apparatus, using the control unit, to treat the subject.

According to still further features in the described preferred embodiments, when the ABI and/or the blood pressure in the affected limb is below a pre-determined value, the control unit is configured to perform at least one safety operation.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are used to designate like elements.

In the drawings:

FIG. 1 provides a cross-sectional view of a sub-atmospheric pressure device of the prior art, the device including an open-cell polymer screen, a flexible hose connecting the foam section to a suction pump, and a flexible polymer sheet overlying the foam-hose assembly to provide the necessary seal;

FIG. 2A provides a cross-sectional view of a sub-atmospheric pressure device of the prior art, the device including a porous screen, an inflatable cuff attached to a semi-rigid cup, and a flexible hose extending from a suction pump to a point within the sealed volume of the cup-cuff assembly;

FIG. 2B provides a side view of a vacuum arrangement of a sub-atmospheric pressure device of the prior art;

FIG. 3 is a block diagram showing the components of a muscle pump stimulation device according to U.S. patent application Ser. No. 11/438,070;

FIG. 4A provides a schematic representation of one aspect of an integrated device or apparatus of the present invention, including both a sub-atmospheric therapy apparatus and a muscle pump device, and

FIG. 4B provides a schematic representation of the integrated device of FIG. 4A, disposed on a limb of a subject.

### DESCRIPTION OF THE PREFERRED EMBODIMENTS

The principles and operation of the treatment method and device of the present invention may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

One aspect of the present invention is a method and device for treating a wound by means of a sub-atmospheric pressure applied in the vicinity thereof, and more particularly, to a treatment method and device utilizing sub-atmospheric pressure therapy in conjunction with a method and device for locally increasing the flow of blood in that vicinity.

It is known to treat skin tissue damage by applying a sub-atmospheric pressure to a wound over an area sufficient to promote development of nutritive blood vessels (granulation tissue) as well as subsequent migration of epithelial cells within the wound area, with the sub-atmospheric pressure being maintained for a time sufficient to facilitate closure of the wound. It has been postulated that wound closure requires that cellular elements of all types migrate from the wound border toward the wound as well as arise from the depths of the wound itself. It has been observed that the use of sub-atmospheric pressure causes increased formation of granulation tissue, a matrix of collagen, fibronectin, and hyaluronic acid carrying macrophages, fibroblasts, and neovasculature. Other understandings of the healing mechanism have been postulated as well.

Without wishing to be bound by theory, it is theorized that stress/tension applied to the wound margins, provided by the use of sub-atmospheric pressure, accelerates healing via the aforementioned mechanisms.

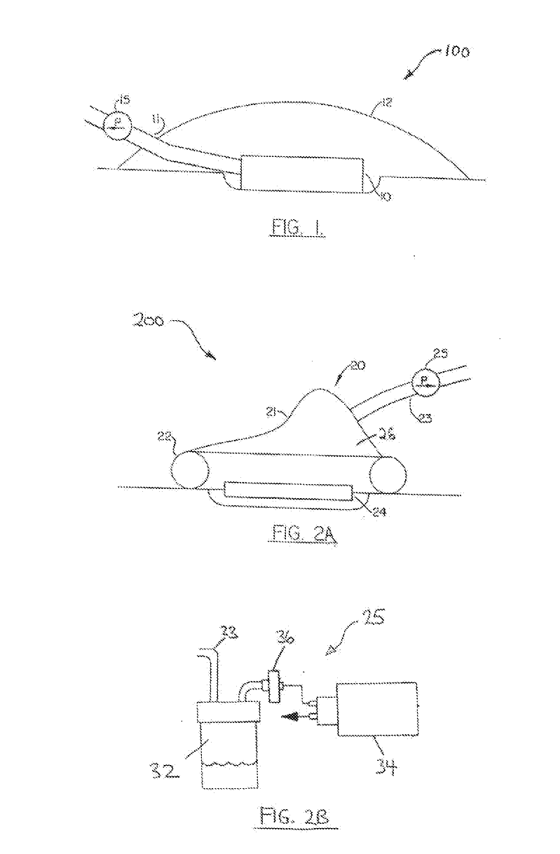

Referring now to the drawings, FIG. 1 is an exemplary embodiment of a sub-atmospheric pressure therapy device 100 as provided by U.S. Pat. No. 5,645,081 to Argenta, et al., which is incorporated by reference for all purposes as if fully set forth herein. Device 100 has a flat open cell polyester foam section 10 sufficiently large to cover a wound 5 and thus prevent wound overgrowth, a flexible hollow tube 11 inserted into foam section 10 and joined thereto with an adhesive, and extending to attach at its opposite end to a suction device such as a vacuum pump 15. Device 100 further includes a covering and sealing arrangement such as an adhesive sheet 12 overlying foam section 10 and tube 11. Adhesive sheet 12 may be adapted to completely surround foam section 10, and may adhere to the skin surrounding wound 5, forming a seal that allows the generation of a sub-atmospheric pressure when vacuum pump 15 operates.

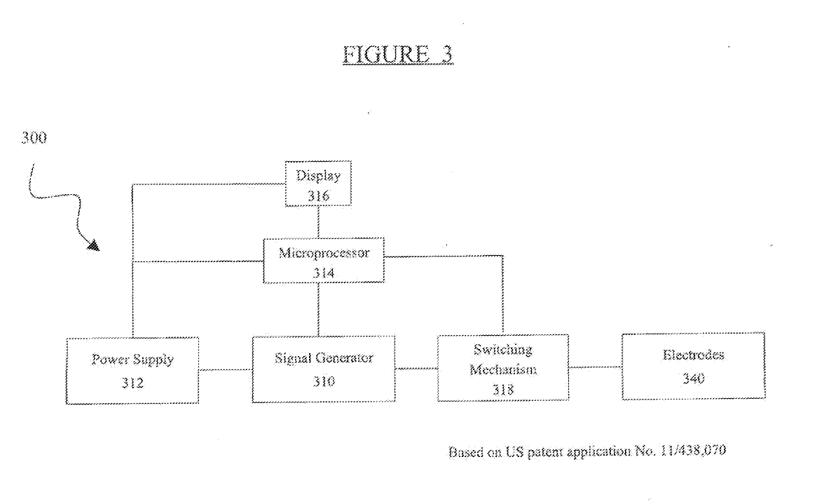

FIG. 2A provides a cross-sectional view of a sub-atmospheric pressure device 200 of the prior art, the device including a porous screen, a cuff such as inflatable cuff 22 attached to a semi-rigid cup 21, and a flexible hose or tubing 23 extending from a suction pump (shown in FIG. 2B) to a point within a sealed volume 26 of the cup-cuff assembly.

Cuff 22 and semi-rigid cup 21 may at least partially define a housing such as an adult cardiopulmonary resuscitation (CPR) mask 20. Inflatable cuff 22 is adapted to contact a skin surface 28. Device 200 further includes an open cell polyester screen 24 overlying wound 5, and a flexible suction hose 23 connected by a tubing connector (not shown) to a vacuum arrangement 25 and extending through a sealed hole 27 in cup 21. Device 200 may be configured such that hose 23 enables fluid communication between cup 21 and a liquid trap bottle 32 (shown in FIG. 2B), to collect any liquid exudate, and between cup 21 and a vacuum pump 34. Device 200 is adapted to be attached over wound 5. After device 200 is attached, suction (e.g., 2-6 pounds vacuum) may be applied. The treatment may significantly accelerate healing and closure of wounds of various types.

As will be apparent to one of ordinary skill in the art, vacuum arrangement 25 may include liquid trap bottle 32, vacuum pump 34, and a vacuum filter 36 coupled between trap bottle 32 and pump 34. Liquid trap bottle 32 may fluidly communicate with a dedicated drainage tube (not shown) to collect liquid in and above wound 5. The sub-atmospheric pressure produced by vacuum arrangement 25 may facilitate drainage, and may facilitate closure of wound 5.

It will be appreciated that there exist many types and variations of sub-atmospheric pressure devices for wound treatment, and that additional types and variations may be conceived by those skilled in the art.

It is generally accepted that the use of such sub-atmospheric pressure devices may be indicated solely for patients having a reasonably healthy local flow of blood in the vicinity of the wound to be treated. As described in greater detail hereinabove, a low ABI index, and more particularly, an ABI index below about 0.7 and/or poor local blood flow as evidenced by a low blood pressure in the ankle, may be a clear contra-indication for using sub-atmospheric pressure therapy. For a vast number of patients having either or both of these conditions, such sub-atmospheric pressure therapy may be largely ineffective, and may also exacerbate the symptoms of ischemia.

The inventive treatment device and method utilize a sub-atmospheric pressure device for treating a wound or skin surface, in conjunction with a method and/or device for locally increasing the flow of blood in the immediate vicinity of the wound or skin surface. By stimulating the local muscle tissue using electrical impulses via the neural motor points (as in functional electrical stimulation devices), an appreciable increase in the local efferent and afferent flow of blood (i.e., in the vicinity of the wound) may be safely achieved, enabling many patients having compromised circulatory status to potentially benefit from the application of sub-atmospheric pressure therapy.

One presently-preferred device and method for effecting the localized increase in blood flow is taught by U.S. patent application Ser. No. 11/438,070, which is incorporated by reference for all purposes as if fully set forth herein.

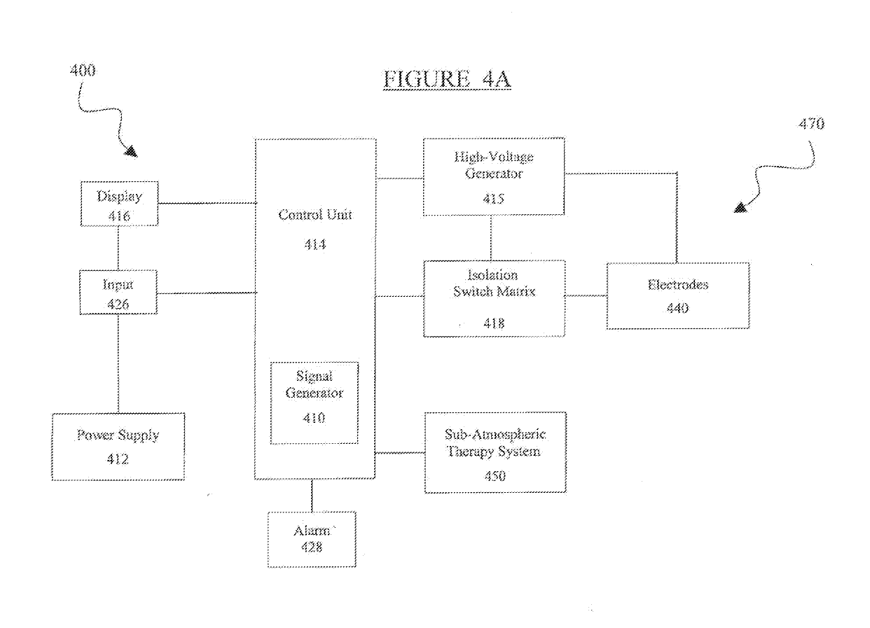

FIG. 3 is a block diagram showing the components of a stimulation device 300 according to U.S. patent application Ser. No. 11/438,070. Signal generator 310 may be operatively connected to a power supply 312. Also connected to power supply 312, may be control unit or microprocessor 314 and display 316. Signal generator 310 may also be integral with microprocessor 314. Signal generator 310 may also operatively connected to a plurality of electrodes 320 via switching mechanism 318. Control unit 314 controls signal generator 310 so as to produce a series of electrical stimulation impulses. These impulses are delivered to electrodes 340, which are adapted to be positioned on a limb or limb segment of the patient. Switching mechanism 318 determines to which pair of electrodes the stimulation impulses will be delivered. Switching mechanism 318 may also be configured as a distributing mechanism that simultaneously distributes a positive or negative signal to two or more electrodes.

Various embodiments of switching mechanism 318 may include a mechanical switching system, an electromechanical relay mechanism, or preferably, an electrical/electronic switching system controlled by control unit 314. A solid state relay having a photo-sensitive metal oxide semiconductor effect transistor (MOSFET) device with an LED to actuate the device is one presently preferred embodiment for switching mechanism 318.

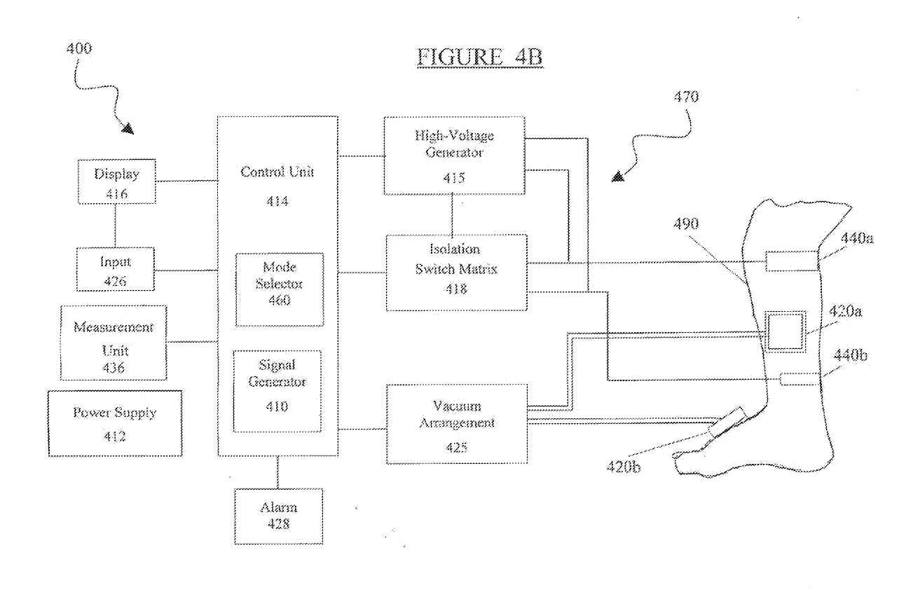

The device and method of the present invention may be better understood with reference to FIGS. 4A and 4B. FIG. 4A provides a schematic representation of one aspect of an integrated device or apparatus 400 of the present invention, including both a sub-atmospheric therapy system or apparatus 450, and a muscle pump or stimulation device 470 that may be similar to stimulation device 300 described hereinabove. FIG. 4B provides a schematic representation of device 400, disposed on a limb of a subject, by way of example, a lower leg. A power supply 412 may provide power to a high-voltage generator 415, a control unit 414, a signal generator 410, a display 416, an input or inputting device 426, an alarm or alarm device 428, a switching mechanism such as an isolation switch matrix 418, and to sub-atmospheric therapy apparatus 450. Control unit 414 may be connected to, or may communicate with, both sub-atmospheric therapy apparatus 450 and various components of stimulation device 470, such as high-voltage generator 415, signal generator 410, display 416, input 426, alarm 428, and switch matrix 418. Electrodes 440 may be connected to control unit 414 via switch matrix 418, or via high-voltage generator 415.

Although signal generator 410, as shown, is disposed within control unit 414, it will be appreciated that signal generator 410 may be disposed outside control unit 414. It will be further appreciated that display 416 and input 426 may serve both sub-atmospheric therapy apparatus 450 and muscle pump or stimulation device 470.

Control unit 414 may be connected to the various controlled components using analog, discrete, and/or serial I/O signals, according to the requirements of the interfaces of the respective components. It will be appreciated that the communication mechanism may include an electronic network of various designs, including serial bus or parallel bus architectures.

Control unit 414 may effect automated control of sub-atmospheric therapy apparatus 450 and stimulation device 470 for a variety of treatment protocols.

With specific reference now to FIG. 4B, a lower leg 490 of a subject has a surface wound, such as a surface wound disposed on the calf, and/or a surface wound disposed on the instep. Each of these surface wounds may be covered by a wound covering and sealing arrangement that includes wound cover and sealing arrangements 420a and 420b, respectively. As described, vacuum arrangement 425 provides suction to the volume defined by a wound cover and sealing arrangement (such as arrangement 420a) and the surface of the limb thereunder, responsive to control unit 414, so as to achieve a sub-atmospheric pressure within that volume.

The efficacy of the sub-atmospheric pressure therapy may be limited by the rate at which arterial blood—containing oxygen, nutrients, white blood cells, and other constituents—is delivered to the area around the wound. For patients having a compromised arterial circulatory status as evidenced by low ABI index and or low ankle blood pressures, such therapy may be of extremely limited value. Additionally, poor venous return and compromised lymphatic drainage contribute to poor healing by preventing “waste products” from being evacuated from the area.

We have found, however, that various mechanical compression methods for locally resolving these concerns to be unsuitable, and possibly deleterious, for use in conjunction with sub-atmospheric pressure therapy, particularly in the case of patients having evidence of arterial, venous and/or lymphatic circulatory compromise. These compression techniques may result in multiple untoward effects including pain, compromise of the circulation, and general reduction in the ability of the patient to engage in normal activities during treatment. By sharp contrast, we have found that by stimulating the local muscle tissue using electrical impulses via the neural motor points, a significant increase in the afferent and efferent flow of blood in the vicinity of the wound may be safely achieved. Without wishing to be bound by theory, we believe that electrically stimulated muscle movement gently and rhythmically alters the configuration of the local blood vessels to increase the local flow of blood. The muscle movement is effected from within the limb, such that skin and surface wound issues are mitigated. In stark contrast, in the various external compression techniques, the driving force—compression—is delivered from outside the surface of the body, such that the skin and wound area lie between the driving force and blood vessels such as deep veins within the limb, and must therefore disadvantageously absorb and transfer the compressive forces.

In one preferred embodiment, the electrical stimulation of the local muscle tissue is performed to effect improved venous return and lymphatic drainage.

In another preferred embodiment, the electrical stimulation of the local muscle tissue is performed to promote at least the local arterial flow.

Stimulation device 470 includes at least two electrodes 440a, 440b adapted to be disposed on the skin surface of the patient. Electrodes 440a, 440b may be fabricated from a conventional conducting foil and a conducting hydrogel adhesive, or from various other conducting medium that will be readily apparent to one of ordinary skill in the art. Various electrodes used in transcutaneous electrical nerve stimulation (TENS) pain reduction devices may be particularly suitable.

While switch matrix 418 may enable the use of at least three, and typically, at least four electrodes, the present invention is capable of operating without such a switch matrix, and with a minimum of two electrodes.

Electrodes 440a, 440b are placed on the skin surface of the subject. The general size, shape, and placement of electrodes 440a, 440b are advantageously determined to achieve superior stimulation of the particular underlying muscles. In the case of lower leg 490 shown in FIG. 4B, the most important underlying muscles include the soleus and gastrocnemius muscles.

Typically, electrodes 440a, 440b may be disposed on either side (e.g. on an upstream side and a downstream side, with respect to the venous return/lymphatic drainage) of the wound, such as on either side of a surface wound covered by cover arrangement 420a. However, an appreciable increase in the flow of blood to the wound area may be achieved even when the wound area is not between the electrodes, and is upstream, with respect to the venous return, from the electrodes. By way of example, electrodes 440a, 440b are both disposed downstream of the wound on the instep, covered by cover arrangement 420b. Upon activating muscle pump 470, venous return and lymphatic drainage may be enhanced, and fluid pressure, and associated pain within the foot may be at least partially alleviated. Furthermore, the stimulated muscles are in the calf, far removed from the instep, such that vigorous contraction of the muscles may be effected without causing discomfort to the instep area.

Thus, according to one aspect of the present invention there is provided an apparatus for facilitating the healing of a wound on a limb of a body of a subject, the apparatus including: (a) a wound treatment assembly having: (i) a wound cover adapted to cover an area above the wound; (ii) a sealing arrangement, associated with the cover, adapted to contact and at least partially seal a volume beneath the cover from an ambient environment; (iii) a vacuum mechanism fluidly communicating with the volume, and adapted to produce a sub-atmospheric pressure within the volume; (b) a muscle contraction device having at least a first electrode and a second electrode, adapted to operatively contact the limb; (c) a control unit, adapted to connect to a power supply and operatively connected to each the electrode, the control unit further adapted to provide, via the electrodes, a sequence of electrical impulses to neural motor points associated with the limb, whereby muscle tissue associated with the neural motor points contracts to effect a localized increase in a flow of blood through a blood vessel in the limb, the control unit being operatively connected to the wound treatment assembly and further adapted to control an operation of the treatment assembly.

The control unit may have a first operating mode enabling a combined treatment protocol including both operation of the wound treatment assembly and operation of the muscle contraction device. The combined treatment protocol may include simultaneous operation of the wound treatment assembly and the muscle contraction device, or including at least intermittent operation of both the wound treatment assembly and the muscle contraction device.

We have found that in many patients, particularly those having a low ABI index, are largely insusceptible to muscle fatigue due to lengthy muscle contraction treatments. This may, in turn, enable lengthy sub-atmospheric treatments of at least 2 to 3 hours, in some cases, at least 6 hours, or even substantially continuously. We have further found that in some cases, the length of the treatment may be extended by intermittently operating the muscle contraction device at a lower than optimal intensity. By doing so, increased blood flow may be sustained over continuous operation (at least 6-24 hours, possibly more), while benefitting from concurrent operation of the sub-atmospheric pressure therapy.

The control unit may advantageously be disposed in a single housing.

The control unit may be configured to implement the combined treatment protocol responsive to a pre-determined sequencing. The control unit may be configured to receive the pre-determined sequencing via an input unit such as input unit 426.

The control unit may have an additional operating mode enabling a treatment protocol including solely operation of the wound treatment assembly, and/or an additional operating mode enabling a treatment protocol including solely operation of the muscle contraction device. The control unit may also have a mode selection switch such as mode selection switch 460, for selecting between the various operating modes.

The control unit may be configured to prompt a user for an ankle-brachial index (ABI) or lower extremity blood pressure of the subject. For example, responsive to an ABI and/or blood pressure below pre-determined values, the control unit may be configured to perform at least one safety operation, including but not limited to producing a warning signal, displaying a recommended treatment protocol, and/or disabling an option of operating the wound treatment assembly without the muscle contraction device. In various circumstances, the control unit may activate alarm 428.

The control unit may be configured to control various parameters pertaining to the vacuum arrangement, including a depth of vacuum produced by the vacuum mechanism. Various other control functions pertaining to the vacuum arrangement will be recognized by one of ordinary skill in the art.

The inventive apparatus may further include a measurement unit 436 adapted to produce at least one measurement of a parameter associated with blood flow in the subject. The control unit may be configured to perform at least one safety operation or other operation responsive to this measurement. Examples of such measurement units include:
blood velocity measurement, e.g., using a Doppler instrument;
on-line ABI measurement (can be input or directly transferred to the control unit), preferably including the identified ankle blood pressure of the affected lower extremity;
RTS (Refill Time Sensor)—measures cyclic changes in the leg (limb) volume due to blood flow (inflow and reflux) using body impedance plethysmography measurements;
MCS (Muscle Contraction Sensor)—measures the magnitude of muscle contraction to provide, inter alia, direct feedback on the physical placement of the electrodes, and effective treatment with reduced user discomfort through the modification of the electrical signal characteristic (e.g. current intensity, pulse train modulation etc.);
LTS (Limb Temperature Sensor)—measures the limb border temperature to provide a clinical treatment indication (e.g., for PAD);
UAS (Ultrasound Artery Sensor)—measures arterial blood flow, e.g., by means of a miniature ultrasonic transducer, to provide quick, direct feedback regarding therapy efficacy.

Using the apparatus and method of the present invention, patients having a characteristically low ABI, below 0.8, below 0.7, below 0.6, below 0.5, and in some cases, as low as about 0.3, may be efficaciously treated with the sub-atmospheric pressure therapy, and substantially without risk, or with significantly reduced risk of infection. In some cases, however, the ABI is not an accurate measurement, and a toe brachial index (TBI) may be used. TBI is a calculation based on the systolic blood pressures of the arm and the systolic blood pressures of the toes. The examination is similar to the ABI except that it is performed with a photoplethysmograph (PPG) infrared light sensor and a small blood pressure cuff placed around the toe. A TBI of 0.8 or greater is considered normal.

As used herein in the specification and in the claims section that follows, the term “limb” is specifically meant to include an arm or a leg. The hand, forearm, upper arm, and shoulder are considered to be parts of a single limb. Similarly, the foot, lower leg, and upper leg are considered to be parts of a single limb.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification, including U.S. Pat. Nos. 5,645,081, 6,458,109, and U.S. Patent Publication No. 20070270917, are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An apparatus for facilitating the healing of a wound on a limb of a body of a subject, the apparatus comprising:
(a) a wound treatment assembly including:
(i) a wound cover adapted to cover an area above the wound;
(ii) a sealing arrangement, associated with said cover, adapted to contact and at least partially seal a volume beneath said cover from an ambient environment;
(iii) a vacuum mechanism fluidly communicating with said volume, and adapted to produce a sub-atmospheric pressure between about 0.01 and 0.95 bar, absolute, within said volume;
(b) a muscle contraction device having at least a first electrode and a second electrode, each said electrode adapted to operatively contact the limb;
(c) a control unit, adapted to connect to a power supply and operatively connected to each said electrode, said control unit further adapted to provide, via said electrodes, a sequence of electrical impulses to neural points associated with the limb, whereby muscle tissue associated with said neural points contracts to effect a localized increase in a flow of blood through a blood vessel in the limb,
said control unit operatively connected to said wound treatment assembly and further adapted to control an operation of said treatment assembly.

2. The apparatus of claim 1, wherein said control unit has at least a first operating mode enabling a combined treatment protocol including both operation of said wound treatment assembly and operation of said muscle contraction device.

3. The apparatus of claim 1, wherein said control unit is configured to implement a combined treatment protocol including simultaneous operation of said wound treatment assembly and said muscle contraction device.

4. The apparatus of claim 1, wherein said control unit is configured to implement a combined treatment protocol including at least intermittent operation of both said wound treatment assembly and said muscle contraction device.

5. The apparatus of claim 4, wherein said control unit is configured to implement said combined treatment protocol responsive to a pre-determined sequencing.

6. The apparatus of claim 5, wherein said control unit is configured to receive said pre-determined sequencing via an input unit associated with said control unit.

7. The apparatus of claim 1, wherein said control unit is configured to prompt a user for an ankle-brachial index (ABI) of the subject and/or an ankle blood pressure.

8. The apparatus of claim 1, wherein said control unit is disposed in a single housing.

9. The apparatus of claim 7, wherein, responsive to an ABI or an ankle blood pressure below pre-determined values, said control unit is configured to perform at least one safety operation.

10. The apparatus of claim 9, wherein said safety operation includes producing a warning signal.

11. The apparatus of claim 9, wherein said safety operation includes displaying a recommended treatment protocol.

12. The apparatus of claim 9, wherein said safety operation includes disabling an option of operating solely said wound treatment assembly.

13. The apparatus of claim 1, wherein said operation of said treatment assembly includes a depth of vacuum produced by said vacuum mechanism.

14. The apparatus of claim 1, further comprising a measurement unit adapted to produce at least one measurement of a parameter associated with blood flow in the subject, said control unit configured to perform at least one safety operation responsive to said measurement.

15. A method of facilitating the healing of the wound, comprising the steps of:
(a) providing the apparatus of claim 1;
(b) disposing said wound cover over the wound;
(c) contacting said sealing arrangement with skin surrounding the wound;
(d) activating said vacuum mechanism to produce said sub-atmospheric pressure within said volume, and
(e) delivering said sequence of electrical impulses, via said electrodes, to effect said localized change in said flow of blood.

16. The method of claim 15, wherein said muscle tissue is activated to effect venous return and lymphatic drainage within the limb.

17. The method of claim 15, wherein said muscle tissue is stimulated to promote a draining of fluid in said muscle tissue, thereby producing said localized increase in said flow of blood.

18. The method of claim 15, further comprising the steps of:
(f) providing said control unit with an ankle-brachial index (ABI) and/or an ankle blood pressure of the desired limb of the subject, and
(g) responsive to said ABI and/or said ankle blood pressure of the desired limb, controlling the apparatus, using said control unit, to treat the subject.

19. The method of claim 18, wherein, when said ABI and/or ankle blood pressure is below a pre-determined value, said control unit is configured to perform at least one safety operation.

20. The method of claim 15, wherein said sub-atmospheric pressure within said volume is between 0.03 bar and 0.3 bar absolute, or between 0.05 and 0.25 bar absolute.
